(19) United States
(12) Patent Application Publication
(10) Pub. No.: US 2014/0235601 A1
(43) Pub. Date: Aug. 21, 2014

(54) Use of Aromatase Inhibitor Or Estrogen Blocker For Increasing Spermatogenesis Or Testosterone Levels In Males

(71) Applicant: Kenneth W. Adams, Oakville (CA)

(72) Inventor: Kenneth W. Adams, Oakville (CA)

(21) Appl. No.: 14/238,014

(22) PCT Filed: Aug. 9, 2012
(86) PCT No.: PCT/CA2012/000746

§ 371 Date: Apr. 11, 2014

Related U.S. Application Data
(60) Provisional application No. 61/521,667, filed on Aug. 9, 2011.

(51) Int. Cl. A61K 31/4196; A61K 45/06

(52) U.S. Cl. CPC A61K 31/4196; A61K 45/06
USPC 514/178; 548/267.4

(57) ABSTRACT

The use of an aromatase blocker or an estrogen blocker is described in a method for increasing spermatogenesis and Seritolli cell function, and/or improving Leydig cell function, in order to to increase endogenous testosterone levels in a male mammal. The levels of active materials used are significantly lower than the levels of these materials used to treat female estrogen sensitive tumors.

### FIELD OF THE INVENTION

The present invention relates to the field of aromatase blockers or estrogen blockers, and in particular, relates to therapeutic agents that can be used to improve testicular Seroti cell function, raise sperm count, improve male fertility, improve testicular Leydig cell function, improve testicular functional capacity, and/or improve/reverse testicular failure in males.

### BACKGROUND OF THE INVENTION

The gonads play an important role in sexual maturation at which time they are responsible for carrying out their function for reproduction. In both sexes the adrenal glands directly and indirectly produce small amounts of testosterone and estrogen. But at puberty, and upon sexual maturation the gonads become the primary source of sex hormone production.

In adult males the testicles produce testosterone and are the site for spermatogenesis, while in adult females the ovaries produce estrogen and are the site of egg production and release. So following puberty, the gonads have the dual role of both gamete production in addition to their role in sex hormone production.

In males, declining testicular function is characterized by not only a drop in both sperm production but also declining testosterone production from the testicles. Adrenal sex hormone production occurs independently and is uncoupled from the pituitary-gonadal axis regulation.

There are many different causes of testicular failure. Testicular failure from genetic causes is relatively rare. One of the most common cause for testicular failure in younger males are undescended testicles in infant males that are not surgically repositioned within the first few months after birth.

Throughout a person's life though, the testicles can be damaged by trauma (blunt trauma, iatrogenic or thermal being the main causes), chemicals or irradiation. For example, pelvic radiation for the treatment of lymphomas, or as a treatment for prostate cancer are universally associated with significant declines in the functional capacity of the testicles to produce sperm and reduced testosterone production.

It would therefore be advantageous to provide a method for improving and/or increasing the functional capacity of the testicles for spermatogenesis. Currently medical science does not identify the role of increased aromatase as a major cause of testicular failure. And giving testosterone to men with low androgen levels worsens this testicular dysfunction.

### SUMMARY OF THE INVENTION

Accordingly, it is a principal advantage of the present invention to provide a method for restoring or enhancing the functional capacity of the testicles for improved sperm production and improved Leydig cell function.

The advantages set out hereinabove, as well as other objects and goals inherent thereto, are at least partially or fully provided by the administration of an aromatase blocker or an estrogen blocker, as set out herein below.

Accordingly, in one aspect, the present invention provides a method for increasing spermatogenesis, and/or improving endogenous testosterone levels in a male mammal, and preferably a human male, by administration of an aromatase blocker or an estrogen blocker.

In a further aspect, the present invention also provides for the use of an aromatase blocker or an estrogen blocker for increasing spermatogenesis, and/or improving Leydig cell function to increase endogenous testosterone levels in a male mammal, and preferably a human male.

### DETAILED DESCRIPTION OF THE INVENTION

The inventor has discovered in males with many different forms of testicular failure, that administration of aromatase blockers or estrogen blockers can significantly restore the functional capacity of the testicles. Clinically this improved functional capacity can result in both:

1. Increased spermatogenesis which is associated with improved Seritoli cell function; and

2. Improved Leydig cell function resulting in increased endogenous testosterone production.

Clinically the inventor has observed that some males with severe testicular failure, who were functionally impotent, had significant increases in sperm counts, improved fertility, improved sexual function, as well as dramatic increases in circulating levels of the sex hormones testosterone and estrogens (and there metabolites), after the administration of an estrogen blocker.

Similarly, the inventor has also observed that some males with severe testicular failure, who were functionally impotent, had significant increases in sperm counts, improved fertility, improved sexual function, as well as dramatic increases in circulating levels of only the sex hormones testosterone (and testosterone metabolites) while estrogen and its metabolites are decreased after the administration of aromatase blocker.

The inventor has further discovered that these effects on spermatogenesis and can be quite significant even when very low levels of aromatase blockers or estrogen blockers are used. In fact in some males, doses as low as 1/100th of the dose of aromatase blockers or estrogen blockers currently being used therapeutically to treat estrogen receptor positive cancer, show positive benefits. Even at these low doses, the use of aromatase blockers can produce significant clinical as well measurably significant biochemical improvements in sperm counts and hormone levels in males.

Without being bound by theory, the inventor theorizes that estrogens inhibit the pituitary gonadal axis and therefore, aromatase blockers or estrogen blockers may increase testicular function by reducing this inhibition.

Accordingly, the present invention involves the use of aromatase blockers, which are preferably delivered as sustained release pellets which have been deposited subcutaneously. Alternatively, these materials may be provided by oral, topical, parenteral, subcutaneous pellet, suppository, sublingual or intranasal administration, or the like.

In the present application, the term “aromatase blocker” refers to those materials which are typically used to “block” or otherwise inhibit, the conversion testosterone into estrogen.

These include, non-selective aromatase blockers such as Aminoglutethimide or Testolactone (Teslac), or selective aromatase blockers such as Anastrozole (Arimidex), Letrozole (Femara), Exemestane (Aromasin), Vorozole (Rivizor), Formestane (Lentaron), Fadrozole (Afema), Chyrisin or the like.

Accordingly, the present invention also involves the use of estrogen blockers, which are preferably delivered as sustained release pellets which have been deposited subcutaneously. Alternatively, these materials may be provided by oral, topical, parenteral, subcutaneous pellet, suppository, sublingual or intranasal administration, or the like.

In the present application, the term “estrogen blocker” refers to those materials which are typically used to “block” or otherwise inhibit, the conversion testosterone into estrogen.

These include, estrogen blockers such as Clomid, Evista, Fareston and Soltamox.or the like.

Combinations of these materials might also be considered, but for clinical and practical reasons aromatase blockers are preferred because the biological effect of estrogen can be easily measured and inferred by measuring estrogen levels, but with an estrogen blocker it is nearly impossible to assess the biological effect of estrogen since estrogen levels will go up as receptors are blocked.

While these materials are all known, the present invention is primarily directed to the use of these materials by male mammals, and preferably human males, in novel applications.

The dose of aromatase blocker is 1/1,000 to 100% of the doses currently recommended for estrogen receptor positive breast cancer. These are doses needed to completely stop all conversion of testosterone into estrogen by completely blocking the aromatase enzyme, for the treatment of estrogen receptor positive breast cancer that can anise in men or women. For milder forms of testicular failure, as commonly occurs in older males, the dose is preferably 1/60th to 1/10th the dose of Femara, Arimdex or other aromatase blocker, needed to completely block the aromatase enzyme (doses typically used for estrogen sensitive tumors).

The dose of estrogen blocker is also 1/1,000 to 100% of the doses currently recommended for estrogen receptor positive breast cancer. The 100% doses is needed to completely block the biological effects of estrogen by completely blocking the estrogen receptor, for the treatment of estrogen receptor positive breast cancer that can arise in men or women. For milder forms of testicular failure, as commonly occurs in older males, the dose of estrogen blocker is preferably 1/60th to 1/10th the dose of estrogen blocker needed to completely block the estrogen receptor (doses typically used for estrogen sensitive tumors).

As such, since typical, prior art treatment levels would be 1 to 5 mg daily of active material, depending on the nature of the active ingredient, the preferred levels of aromatase blocker or estrogen blocker treatments in males, in the present application, would be between 0.001 and 5 mg daily, and more preferred treatment levels would be between 0.167 and 0.5 mg. Still more preferably, the treatment levels would be between 0.250 and 0.400 mg daily, based on the normal dosages currently recommended for estrogen receptor positive breast cancer. For more severe forms of testicular failure higher doses may be required.

Typically, the level of aromatase blocker or estrogen blocker is preferably determined based on individuals clinical response. The clinical response to be titrated may be sperm count when treating infertility, but when erectile dysfunction and low libido are the males primary concern, then the dose is titrated based on libido (which is related to the estrogen and testosterone levels).

### EXAMPLES

### Clinical Example A

A 29 year old married male with a history of undescended testicles that were surgically corrected at the age of three. This man presented to the inventor with small very atrophic testicles, impotence (difficulty in erecting, only occasionally able to have successful intercourse only with PDE5 inhibitors) and an inability to conceive despite two years of unprotected sexual intercourse with his wife.

Following treatment with ⅛mg (e.g. ⅛th of a 1 mg tablet, or 0.125 mg) of Arimidex daily patient experienced a dramatic increase in sperm counts, significant increase in testosterone and estrogen levels, and dramatic improvement in erectile function. And the patient ultimately was able to conceive and have a child. All of this is a result of the improved testicular function that this relatively low dose of Arimidex provided for this patient.

### Clinical Example B

A 23 year athletic and muscular male complaining of low libido, erectile dysfunction and ejaculatory failure. Patient was needing to use Viagra in order to function sexually, and had no desire for sex. Sex hormones testosterone and estrogen were measured at prepubertal levels. The testicles were extremely small and atrophic, and a prior testicular biopsy showed a complete absence of spermatogenesis.

Upon starting the aromatase blocker Femara at a dose of ⅛ to ½ of a 2.5 mg tablet, this patient had a dramatic increase in testicular function with restoration of sex drive, improved erections, restoration of ejaculations during intercourse and testosterone levels were restored to the high supraphysiologic levels.

### Clinical Example C

A sexually active 68 year old male with biopsy confirmed prostate cancer elects to treat his cancer with pelvic radiation. Following radiation there is a progressive decline in testosterone levels, combined with testicular atrophy and increasing erectile dysfunction. The patient received 1/40th of a 2.5 mg Letrazole tablet daily and experienced a significant improvement in testicular function, as evidenced by a dramatic rise in testosterone levels.

Thus, it is apparent that there has been provided, in accordance with the present invention, a method and use which fully satisfies the goals, objects, and advantages set forth hereinbefore. Therefore, having described specific embodiments of the present invention, it will be understood that alternatives, modifications and variations thereof may be suggested to those skilled in the art, and that it is intended that the present specification embrace all such alternatives, modifications and variations as fall within the scope of the appended claims.

Additionally, for clarity and unless otherwise stated, the word “comprise” and variations of the word such as “comprising” and “comprises”, when used in the description and claims of the present specification, is not intended to exclude other additives, components, integers or steps. Further, the invention illustratively disclosed herein suitably may be practiced in the absence of any element which is not specifically disclosed herein.

Moreover, the words “substantially” or “essentially”, when used with an adjective or adverb is intended to enhance the scope of the particular characteristic; e.g., substantially planar is intended to mean planar, nearly planar and/or exhibiting characteristics associated with a planar element.

Also, while this discussion has addressed prior art known to the inventor, it is not an admission that all art discussed is citable against the present application.

1. A method for increasing spermatogenesis and Seritolli cell function and/or improving Leydig cell function to increase endogenous testosterone levels in a male mammal, by an administration of an aromatase blocker or estrogen blocker, or combination thereof.

2. A method as claimed in claim 1 wherein said aromatase blocker is selected from the group consisting of Aminoglutethimide, Testolactone (Teslac), Anastrozole (Arimidex), Letrozole (Femara), Exemestane (Aromasin), Vorozole (Rivizor), Formestane (Lentaron) and Fadrozole (Afema), or combinations thereof.

3. A method as claimed in claim 1 wherein said estrogen blocker is selected from the group consisting of Clomid, Evista, Fareston and Soltamox, or combinations thereof.

4. A method as claimed in claim 1 wherein said male mammal is a human male.

5. A method as claimed in claim 1 wherein the level of aromatase blocker or estrogen blocker is between 1/1,000th and 100% of the level of aromatase blocker and/or estrogen blocker used to treat estrogen receptor positive breast tumors or female estrogen sensitive tumors.

6. A method as claimed in claim 1 wherein the level of aromatase blocker or estrogen blocker is between 0.001 and 5 mg daily, of active material.

7. A method as claimed in claim 1 wherein the level of aromatase blocker or estrogen blocker is between 0.250 and 0.400 mg daily, of active material.

8. Use of an aromatase blocker or an estrogen blocker for increasing spermatogenesis, and/or improving endogenous testosterone levels in a male mammal.

9. Use as claimed in claim 8 wherein said aromatase blocker is selected from the group consisting of Aminoglutethimide, Testolactone (Teslac), Anastrozole (Arimidex), Letrozole (Femara), Exemestane (Aromasin), Vorozole (Rivizor), Formestane (Lentaron) and Fadrozole (Afema), or combinations thereof.

10. Use as claimed in claim 8 wherein said estrogen blocker is selected from the group consisting of Clomid, Evista, Fareston and Soltamox, or combinations thereof.

11. Use as claimed in claim 8 wherein said male is a human male.

12. Use as claimed in claim 8 wherein the level of aromatase blocker or estrogen blocker is between 1/1,000th and 100% of the level of aromatase blocker used to treat female estrogen sensitive tumors or female estrogen sensitive tumors.

13. Use as claimed in claim 8 wherein the level of aromatase blocker or estrogen blocker is between 0.001 and 5 mg daily, of active material.

14. Use as claimed in claim 8 wherein the level of aromatase blocker or estrogen blocker is between 0.250 and 0.400 mg daily, of active material.
